(19) United States
(12) Patent Application Publication
(10) Pub. No.: US 2019/0234209 A1
(43) Pub. Date: Aug. 1, 2019

(54) MEASURING FLUID DENSITY IN A FLUID FLOW

(71) Applicant: Saudi Arabian Oil Company, Dhahran (SA)

(72) Inventors: Chidirim Enoch Ejim, Dhahran (SA); Jinjiang Xiao, Dhahran (SA)

(73) Assignee: Saudi Arabian Oil Company, Dhahran (SA)

(21) Appl. No.: 15/883,905

(22) Filed: Jan. 30, 2018

(51) Int. Cl. E21B 49/08; G01N 33/28; G01N 9/36; E21B 47/06

(52) U.S. Cl. CPC E21B 49/08; G01N 33/2823; G01N 33/2847; E21B 43/128; E21B 47/06; E21B 2049/085; G01N 9/36

(57) ABSTRACT

A downhole production system includes an electric submersible pump (ESP) configured to be positioned within a wellbore. A measurement device is positioned upstream of an ESP suction. The measurement device is fluidically coupled to the suction of the ESP. The measurement device is configured to measure a water-cut of a production fluid flowing into the ESP. The measurement device includes a first flow section configured to receive a fluid flow, a second flow section positioned downstream of the first flow section which is fluidically coupled to the first flow section and at an angle from the first section, a first differential pressure sub-system positioned within the first flow section and a second differential pressure sub-system positioned within the second flow section, downstream of the first differential pressure sub-system. A packer is positioned uphole of a fluid inlet of the measurement device and is configured to direct fluid into the measurement device.

### TECHNICAL FIELD

This disclosure relates to measuring properties of flowing fluids.

### BACKGROUND

In hydrocarbon production, a producing well can produce both hydrocarbons and water. Knowing the ratio of water to hydrocarbons is important for determining how much hydrocarbons a well produces, as well as running flow assurance calculations. Two types of measurement tools used to determine a downhole water content of a production flow are based on technology found in a gamma ray densitometer and a gradiomanometer. The gamma ray tool is based on the principle that the absorbance of gamma rays is inversely proportional to the density of the medium through which the gamma rays pass. Such a tool includes a gamma ray source, a channel through which the fluid medium can flow through, and a gamma ray detector. The gradiomanometer is a device used to determine average fluid density by measuring the pressure difference between two pressure sensors. The pressure sensors are typically spaced (axially) about 0.6m (2 feet) from each other.

In some instances, an electric submersible pump can be installed within a completed well to increase production rates.

### SUMMARY

This disclosure describes technologies relating to measuring fluid density in a fluid flow.

In a first example aspect, a downhole production system includes an electric submersible pump configured to be positioned within a wellbore. A measurement device is positioned upstream of an electric submersible pump suction. The measurement device is fluidically coupled to the suction of the electric submersible pump. The measurement device is configured to measure a water-cut of a production fluid flowing into the electric submersible pump. The measurement device includes a first flow section configured to receive a fluid flow, a second flow section positioned downstream of the first flow section which is fluidically coupled to the first flow section and at an angle from the first section, a first differential pressure sub-system positioned within the first flow section and a second differential pressure sub-system positioned within the second flow section, downstream of the first differential pressure sub-system. A packer is positioned uphole of a fluid inlet of the measurement device and is configured to direct fluid into the measurement device.

In another aspect, combinable with any of the other aspects, the system includes a shroud that envelopes and encloses an uphole end of the measurement device and the electric submersible pump suction. The shroud is configured to enclose a fluid flow and direct the fluid flow from the measurement device and into the electric submersible pump suction.

In another aspect, combinable with any of the other aspects, the packer is a first packer. The system includes a second packer located uphole of the electric submersible pump and configured to direct a fluid flow from the measurement device and into the electric submersible pump suction.

In another aspect, combinable with any of the other aspects, the system includes a controller configured to determine a density of the fluid flow using Equation 1 (described later) and determine a water-cut using Equation 2 (described later).

In another aspect, combinable with any of the other aspects, the first differential pressure sub-system includes a first pressure sensor and a second pressure sensor. The second differential pressure sub-system includes a third pressure sensor and a fourth pressure sensor.

In a second example aspect, a fully developed, multi-phase fluid flow is received into a measurement device. The fluid flow is directed through a first flow passage of the measurement device. A pressure drop of the fully developed, multi-phase fluid flow is measured within the first flow passage. The fluid flow from the first flow passage is directed to a second flow passage of the measurement device. The second flow passage is fluidically coupled to the first flow passage and is at an angle from the first flow passage. A pressure drop of the fully developed, multi-phase flow is measured within the second flow passage. A density of the fluid flow is determined in response to measuring the pressure drop within the first flow passage and the pressure drop within the second flow passage.

In another aspect, combinable with any of the other aspects, a density of the fluid flow is determined using Equation 1 (described later).

In another aspect, combinable with any of the other aspects, the water-cut is determined using Equation 2 (described later).

In a third example aspect, a measurement device includes a first flow passage configured to receive a fully developed, multi-phase fluid flow. The measurement device includes a second flow passage configured to receive the fully developed, multi-phase fluid flow. The second flow passage is positioned downstream of the first flow passage, is fluidically coupled to the first flow passage, and is at an angle from the first flow passage. The measurement device includes a first pressure sensor positioned within the first flow passage and configured to measure a first pressure within the first flow passage. The measurement device includes a second pressure sensor positioned within the first flow passage, downstream of the first flow sensor, and configured to measure a second pressure within the first flow passage. The measurement device includes a third pressure sensor positioned within the second flow passage and configured to measure a third pressure within the second flow passage. The measurement device includes a fourth pressure sensor positioned within the second flow passage, downstream of the third pressure sensor, and configured to measure a fourth pressure within the second flow passage. The measurement device includes a controller configured to receive a first pressure signal from the first pressure sensor, a second pressure signal from the second pressure sensor, a third pressure signal from the third pressure sensor, and a fourth pressure signal from the fourth pressure sensor, and configured to determine a density of the fully developed, multi-phase fluid flow at least in part based on the first signal, the second signal, the third signal, and the fourth signal.

In another aspect, combinable with any of the other aspects, the first flow passage and the second flow passage have the same cross-sectional area.

In another aspect, combinable with any of the other aspects, a distance between the first pressure sensor and an entrance to the first flow passage is at least five effective diameters of the first flow passage.

In another aspect, combinable with any of the other aspects, a distance between the second pressure sensor and the second flow passage is at least five effective diameters of the first flow passage.

In another aspect, combinable with any of the other aspects, a distance between the third pressure sensor and the first flow passage is at least five effective diameters of the first flow passage.

In another aspect, combinable with any of the other aspects, a distance between the fourth pressure sensor and a downstream obstruction is at least five effective diameters of the second flow passage.

In another aspect, combinable with any of the other aspects, a distance between the first pressure sensor and the second pressure sensor is at least five effective diameters of the first flow passage.

In another aspect, combinable with any of the other aspects, a distance between the third pressure sensor and the second pressure sensor is at least ten effective diameters of the second flow passage.

In another aspect, combinable with any of the other aspects, the angle between the first flow passage and the second flow passage is substantially within five to seven degrees.

In another aspect, combinable with any of the other aspects, a length of the first flow passage and a length of the second flow passage are substantially within fifteen and twenty effective diameters of the respective flow passages.

In another aspect, combinable with any of the other aspects, a length of the first flow passage is different from a length of the second flow passage.

In another aspect, combinable with any of the other aspects, the measurement device is configured to be installed upstream of an electric submersible pump.

The details of one or more implementations of the subject matter described in this disclosure are set forth in the accompanying drawings and the description. Other features, aspects, and advantages of the subject matter will become apparent from the description, the drawings, and the claims.

### BRIEF DESCRIPTION OF THE DRAWINGS

FIG. 1A is a side, half-cross sectional view of an example density measuring device.

FIGS. 1B-1C are side, half-cross sectional views of example differential pressure systems.

FIG. 2 is a side, half-cross sectional view of an example downhole production system installed within a wellbore.

FIG. 3 is a side, half-cross sectional view of an example downhole production system installed within a wellbore.

FIG. 4 is a flowchart of an example method that can be utilized with aspects of this disclosure.

Like reference numbers and designations in the various drawings indicate like elements.

### DETAILED DESCRIPTION

Production of oil-water mixtures is very common in oilfield operations. One of the physical properties of the fluid mixture required by production engineers, reservoir engineers, or the field operators is the water-cut of the produced fluid downhole. Water-cut is the ratio of water volume flow rate to the oil-water (mixture) volume flow rate. To determine the water-cut production, accurate knowledge of the downhole oil-water mixture density is needed.

Measurement tools, such as a gamma ray tool, have low sensitivity in oil-water flows, a small sampling size, and a statistical nature of the measurement due to fluctuations in the readings that are inherent in any nuclear measurement. In addition, the use of nuclear-based technology can cause health, safety, security, and environment concerns, which can be prohibitive in some operator jurisdictions.

Measurement tools, such as a gradiomanometer, have measurements affected by well deviation, kinetic effect, and friction effect. Well deviation is the orientation of the well trajectory from vertical. The kinetic effect occurs from a difference in fluid velocity at the upper and lower sensor locations, which is typically caused by a change in geometry at the respective locations. The friction effect is due to pressure losses resulting from tool surface friction and depends on tool geometry and flow rate. During density measurements using a gradiomanometer, only the pressure difference measurements and well deviation corrections are used to determine the average fluid density. The friction effect is typically neglected for flow rates below 2000 barrels per day (BPD). Neglecting the friction term affects the gradiomanometer's accuracy. For flow rates above 2000 BPD, where the frictional term is not neglected, determining the mixture density requires additional equipment. First, another device needs to be installed to determine the mixture flow rate. Second, there is a complex relationship between the mixture flow rate, mixture density, and frictional term. A further limitation of the gradiomanometer is the degree of well deviation in which they are used. Measurements are not valid in horizontal wells, and as such, use of these tools are restricted only to vertical or inclined wells.

This disclosure describes an apparatus and method for measuring the density of oil-water mixtures and determining an oil-to-water ratio during production operations either downhole or topside. The device includes a tubular pipe with two flow sections. The first flow section is at a first angle from horizontal and the second flow section is at a second angle from horizontal. In some instances, the first angle and the second angle can be measured from the longitudinal axis of the well with identical results. Both flow sections are fluidically connected to one another and have substantially the same cross-sectional flow area, that is, the flow areas are identical with variations falling within machining tolerances. A pressure drop is measured within each section. A density and water-cut are determined based on the measured pressure drops. The measurement device can be used in a vertical, deviated, or horizontal well. In some instances, the measurement device can be used at a topside facility.

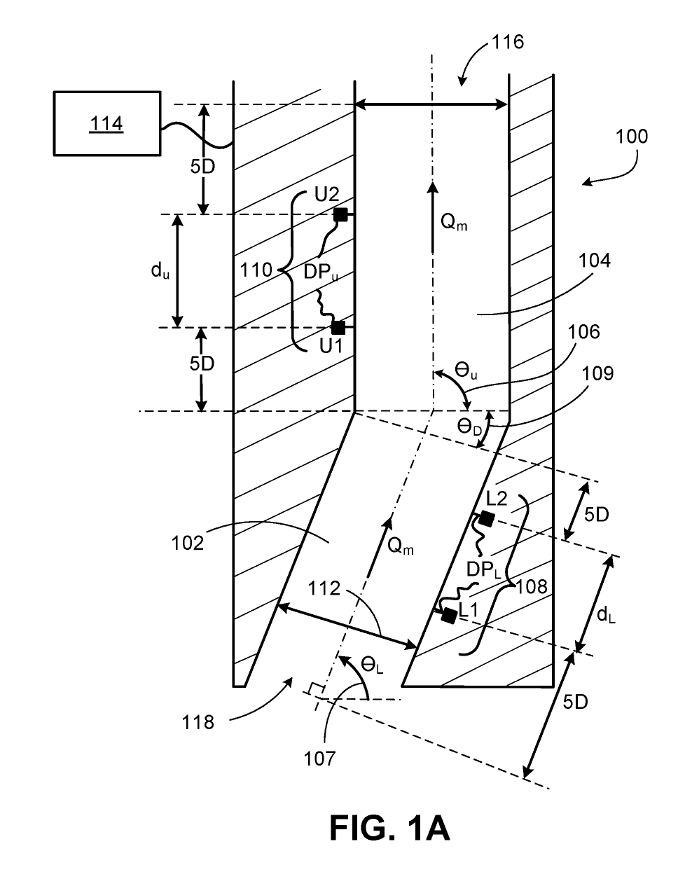

FIG. 1A shows a side half-cross-sectional view of an example measurement device 100. In some implementations, the measurement device 100 is configured to be installed upstream of an electric submersible pump (ESP). In some implementations, the measurement device is configured to be installed downstream of an ESP, for example, at a topside facility. The measurement device 100 includes a first flow passage 102 configured to receive a fully developed, multi-phase fluid flow, for example, from a wellbore. In some instances, the first flow passage 102 is of sufficient length for the fluid flow, at a specified fluid flow rate, to become fully developed within the first flow passage 102. Fully developed flow, in the context of this disclosure, is when the viscous effects, due to the shear stress between the fluid particles and pipe wall, create a fully developed velocity profile for a fluid as it travels through the length of a straight pipe. The measurement device also includes a second flow passage 104 configured to receive the fully developed, multi-phase fluid flow from the first flow passage 102. In some instances, the second flow passage 104 is of sufficient length for the fluid flow, at a specified fluid flow rate, to become fully developed within the second flow passage 104. The second flow passage 104 is positioned downstream of the first flow passage 102 and is fluidically coupled to the first flow passage 102. That is, an outlet of the first flow passage 102 is directly connected to an inlet of the second flow passage 104 such that fluid exiting the outlet of the first flow passage 102 flows into the inlet of the second flow passage 104.

The second flow passage 104 defines an angle 106 (θ_{U}) which is measured from the longitudinal axis of the second flow passage 104 and a horizontal line, that is, a line parallel to a surface of the wellbore in which the measurement device 100 is positioned. The first flow passage 102 defines an angle 107 (θ_{L}) which is measured from the longitudinal axis of the first flow passage 102 and the horizontal line. In some implementations, a difference between the angle 106 and the angle 107, that is, the angle 109 (θ_{D}), is substantially within five to seven degrees (within machining tolerances). In some implementations, the first flow passage 102 and the second flow passage 104 have the same cross-sectional area. In some instances, a length of the first flow passage 102 and a length of the second flow passage 104 are substantially within fifteen to twenty effective diameters of the respective flow passages. That is, the length of each flow passage is fifteen to twenty times the diameter of the respective flow passage. In some instances, a length of the first flow passage 102 is different from a length of the second flow passage 104. In some instances, a length of the first flow passage 102 is substantially the same (within standard machining tolerances) as a length of the second flow passage 104. In some instances, the first flow passage 102 and the second flow passage 104 are both of sufficient length for the fluid flow, at a specified fluid flow rate, to become fully developed within both the first flow passage 102 and the second flow passage 104. For example, the flow can become fully developed within five pipe diameters from the inlet of either the first flow passage 102 or the second flow passage 104.

Both the first flow passage 102 and the second flow passage 104 include a differential pressure sub-system. For example, a first differential pressure sub-system 108 is positioned within the first flow passage 102, and a second differential pressure sub-system 110 is positioned within the second flow passage 104, downstream of the first differential pressure sub-system 108. In some implementations, the measurement device 100 can include a controller 114 configured to receive a signal from each of the first differential pressure sub-system 108 and the second differential sub-system 110. Details on the operations of the controller 114 are explained in greater detail later in this disclosure.

In some instances, illustrated in FIGS. 1B-1C, either the first differential pressure sub-system 108 (FIG. 1A), the second differential pressure sub-system 110 (FIG. 1A), or both, can include a single pressure differential sensor 150 connected via a first sensor flow passage 152a and a second sensor flow passage 152b, each passage leading to a main flow passage 158, such as the first flow passage 102 or the second flow passage 104 shown in FIG. 1A. In such an implementation, a minimum distance 154 is used to ensure that the flow at the entrance of the second sensor flow passage 152b is sufficiently far downstream of the first sensor flow passage 152a so that both of the sensor flow passages experience fully developed flow within the main flow passage 158. For example, in some implementations, a distance between the first sensor flow passage 152a and the second sensor flow passage 152b is at least five pipe diameters in length. That is, the distance between the first sensor flow passage 152a and the second sensor flow passage 152b is at least five times that of a flow path diameter 112 (FIG. 1A). In some implementations, the controller 114 is configured to receive a signal from a first differential pressure sensor positioned in the first flow passage 102 and a second pressure differential sensor in the second flow passage 104.

In some implementations, such as the implementation shown in FIG. 1C, a first pressure sensor 156a is positioned within the main flow passage 158. The first pressure sensor 156a is configured to measure a first pressure within the main flow passage 158. In such an implementation, a second pressure sensor 156b is positioned within the main flow passage 158 downstream of the first pressure sensor 156a. The second pressure sensor 156b is configured to measure a second pressure within the main flow passage 158. In such an implementation, the minimum distance 154 is used to ensure that the flow at the entrance of the second pressure sensor 156b is sufficiently far downstream of the first pressure sensor 156a so that both of the sensors experience fully developed flow. For example, in some implementations, a distance between the first pressure sensor 156a and the second pressure sensor 156b is at least five pipe diameters in length. That is, the distance between the first pressure sensor 156a and the second pressure sensor 156b is at least five times that of a flow path diameter 112 (FIG. 1A). In some implementations, when individual flow sensors are used, the first pressure sensor 156a and the second pressure sensor 156b are positioned in the first flow passage 102, while a third pressure sensor and a fourth pressure sensor are positioned in the second flow passage 104. In such an implementation, the third pressure sensor is configured to measure a third pressure within the second flow passage 104, while the fourth pressure sensor, which is downstream of the third pressure sensor, is configured to measure a fourth pressure within the second flow passage 104. In such an instance, the controller is configured to receive a first pressure signal from the first pressure sensor, a second pressure signal from the second pressure sensor, a third pressure signal from the third pressure sensor, and a fourth pressure signal from the fourth pressure sensor, to determine a differential pressure between the first and second sensor, as well as a differential pressure between the third and fourth sensor.

The spacing between various flow interfering components, such as an entrance 118 (FIG. 1A) of the measuring device 100 (FIG. 1A), can have a measurable effect on pressures at various points. As a result, spacing can be a design constraint within the measurement device 100 (FIG. 1A). For example, in some instances a distance between the first pressure sensor 156a (or first sensor flow passage 152a) and the entrance 118 (FIG. 1A) to the first flow passage 102 (FIG. 1A) is at least five effective diameters of the first flow passage 102. In some instances, a distance between the second pressure sensor 156b (or the second sensor flow passage 152b) and first entrance to the second flow passage 104 (FIG. 1A) is at least five effective diameters of the first flow passage 102 (FIG. 1A). In some instances, a distance between a third pressure sensor (or a third sensor flow passage) and entrance to the second flow passage 104 (FIG. 1A) is at least five effective diameters of the second flow passage 104 (FIG. 1A) In some instances, a distance between a fourth pressure sensor (or a fourth flow sensor passage) and a downstream obstruction, such as an outlet 116 (FIG. 1A) to the measurement device 100 (FIG. 1A), is at least five effective diameters of the second flow passage 104 (FIG. 1A).

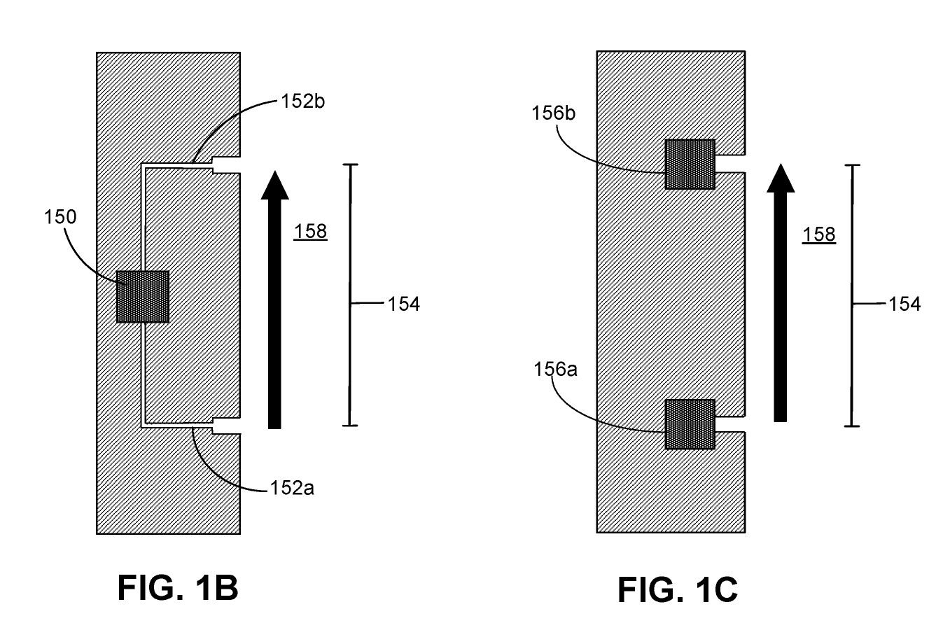

As previously discussed, the measurement device 100 can be installed downhole with an ESP 200. FIG. 2 is a half cross-sectional view of an example of such an implementation. The ESP 200 is configured to be positioned within a wellbore 202 and includes a pumping section 206, protector 207, ESP monitoring sub 209 and a motor 212 configured to power the pumping section 206. In the illustrated implementation, the measurement device 100 is positioned upstream (downhole) of an ESP suction 204. The measurement device 100 is fluidically coupled to the suction 204 of an ESP pumping section 206. In such an implementation, the measurement device 100 measures a water-cut of a production fluid flowing into the ESP 200. A packer 208 can be secured to the measurement device 100 and directs the production fluid into the measurement device 100. That is, the packer 208 is positioned uphole of the entrance 118 of the measurement device 100 and downhole of the exit 116 of the measurement device 100.

In the illustrated implementation, a shroud 210 envelopes and encloses the exit 116 (uphole end) of the measurement device 100 and the ESP suction 204. The shroud 210 is configured to enclose a fluid flow and direct the fluid flow from the measurement device 100 and into the electric submersible pump suction 204.

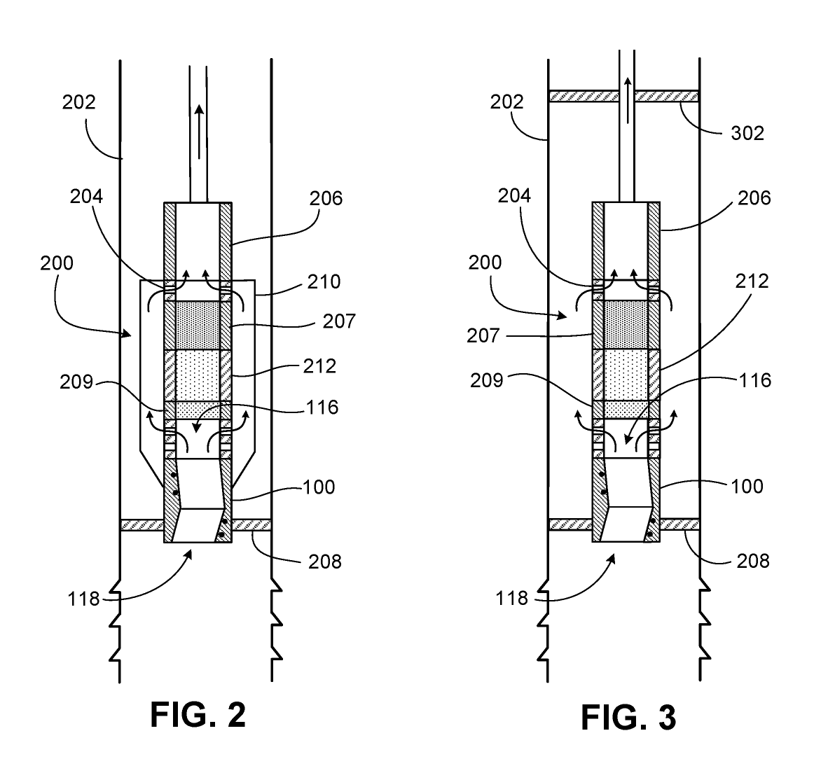

FIG. 3 is a half cross-sectional view of an example implementation of the measurement device 100 being used in conjunction with an ESP 200. Rather than the shroud 210, the illustrated implementation uses a second packer 302 located uphole of the ESP 200. The second packer 302 is configured to direct a fluid flow from the measurement device 100 into the electric submersible pump suction 204.

In some implementations, when the measurement device 100 includes a controller 114, the controller 114 is configured to determine a density of the fully developed, multi-phase fluid flow at least in part based on the signals from the first differential pressure sub-system 108 and the second pressure differential sub-system 110.

The controller 114 can be located either downhole or at a topside facility. The controller 114 includes one or more processors and non-transitory memory storing computer instructions executable by the one or more processors to perform operations, for example, the operations to determine density. Alternatively, or in addition, the controller 114 can be implemented as processing circuitry, including electrical or electronic components (or both), configured to perform the operations described here. The controller 114 is configured to determine a density of the fluid flow using the following equation:

$${ρ}_{m} = \frac{\frac{{DP}_{U}}{{d}_{U}} - \frac{{DP}_{L}}{{d}_{L}}}{g ( Sin {θ}_{U} - Sin {θ}_{L} )} \qquad ( Eq . 1 )$$

where DP_{L} is a pressure drop within the first flow section 102, DP_{U} is the pressure drop within the second flow section 104, d_{L} is an axial distance between two pressure measurement locations of the first flow section 102, d_{U} is an axial distance between two pressure measurement locations of the second flow section 104, g is an acceleration due to gravity, θ_{L} is an inclination angle of a flow axis of the first flow section 102 from horizontal, θ_{U} is an inclination angle of the second flow section 104 from horizontal, and ρₘ is a density of the fluid flow. Once density of the fluid flow is determined, then, the controller 114 is configured to determine a water-cut using the following equation:

$$WC = \frac{{ρ}_{m} - {ρ}_{o}}{{ρ}_{w} - {ρ}_{o}} \qquad ( Eq . 2 )$$

where ρₒ is a density of an oil portion of the fluid flow, ρ_{w} is a water density of the fluid flow, and WC is the water-cut. The oil density variation with temperature and pressure would have been obtained with pressure-volume-temperature (PVT) analysis on the hydrocarbon obtained in the early life of the well. In the operation of the flow meter, the downhole pressure and temperature can be obtained from the monitoring sub 209. Based on the temperature and pressure, the density of the pure oil can be determined and can be used in Equation 2. Density of water can be determined by the controller 114 based on the pressure and temperature of the fluid flowing through the measurement device 100.

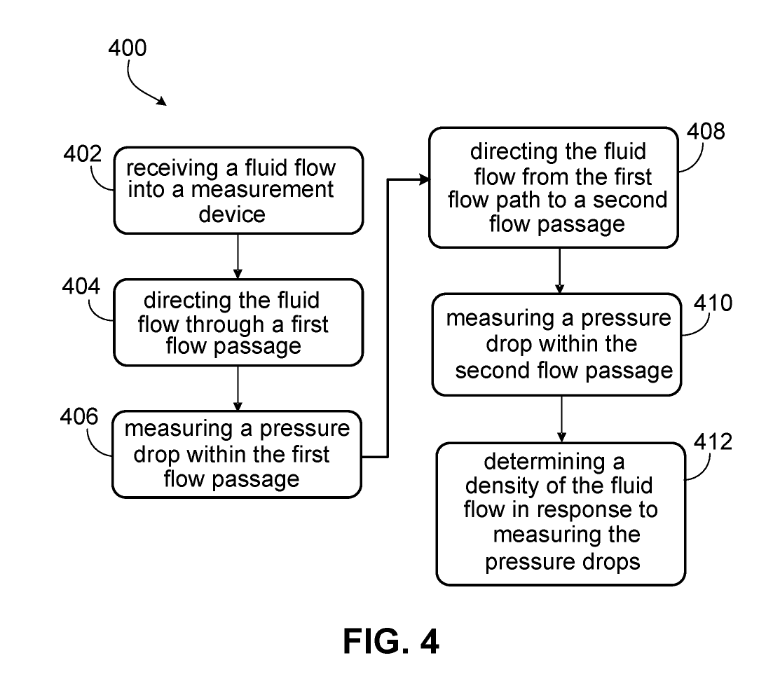

FIG. 4 is a flowchart of an example method 400 that can be used with aspects of this disclosure. At 402, a fully developed, multi-phase fluid flow is received into the measurement device 100. At 404, the fluid flow is directed through a first flow passage 102 of the measurement device 100. At 406, a pressure drop of the fully developed, multi-phase fluid flow within the first flow passage 102 is measured. At 408, the fluid flow is directed from the first flow passage 102 to the second flow passage 104 of the measurement device 100. The second flow passage 104 is fluidically coupled to the first flow passage 102, and the second flow passage 104 is at an angle from the first flow passage 102. At 410, a pressure drop of the fully developed, multi-phase fluid flow within the second flow passage 104 is measured. At 412, a density of the fluid flow is determined in response to measuring the pressure drop within the first flow passage 102 and the pressure drop within the second flow passage 104. In some implementations, the density of the fluid flow is determined by using Eq. 1. In some instances, a water-cut of the fluid flow is determined in response to determining the density of the fluid flow. In some instances, Eq. 2 is used to determine the water-cut.

While this disclosure contains many specific implementation details, these should not be construed as limitations on the scope of what may be claimed, but rather as descriptions of features specific to particular implementations. Certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the implementations described above should not be understood as requiring such separation in all implementations, and it should be understood that the described components and systems can generally be integrated together in a single product or packaged into multiple products. For example, the measurement device 100 can be positioned downstream of the ESP 200, or at a topside facility.

Thus, particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. Certain implementations described in this disclosure are based on the layout of conventional tubing deployed ESP systems. In some implementations, the installation of the meter can be modified to fit a tubing deployed inverted ESP system as well as a cable deployed ESP system. In such implementations, the meter can be positioned downstream of the pumping section of the ESP.

What is claimed is:

1. A downhole production system comprising:
an electric submersible pump configured to be positioned within a wellbore;
a measurement device positioned upstream of an electric submersible pump suction, the measurement device being fluidically coupled to the suction of the electric submersible pump, the measurement device configured to measure a water-cut of a production fluid flowing into the electric submersible pump the measurement device comprising:
a first flow section configured to receive a fluid flow;
a second flow section positioned downstream of the first flow section, the second flow section fluidically coupled to the first flow section, the second flow section being at an angle from the first section;
a first differential pressure sub-system positioned within the first flow section;
a second differential pressure sub-system positioned within the second flow section, downstream of the first differential pressure sub-system; and
a packer positioned uphole of a fluid inlet of the measurement device, the packer configured to direct fluid into the measurement device.

2. The downhole production system of claim 1, further comprising:
a shroud that envelopes and encloses an uphole end of the measurement device and the electric submersible pump suction, the shroud configured to enclose a fluid flow and direct the fluid flow from the measurement device and into the electric submersible pump suction.

3. The downhole production system of claim 1, wherein the packer is a first packer, the downhole production system further comprising:
a second packer located uphole of the electric submersible pump, the second packer configured to direct a fluid flow from the measurement device and into the electric submersible pump suction.

4. The downhole production system of claim 1, further comprising a controller configured to:
determine a density of the fluid flow using the following equation:
$${ρ}_{m} = \frac{\frac{{DP}_{U}}{{d}_{U}} - \frac{{DP}_{L}}{{d}_{L}}}{g ( Sin {θ}_{U} - Sin {θ}_{L} )}$$
where DP_{L} is a pressure drop within the first flow section, DP_{U} is the pressure drop within the second flow section, d_{L} is an axial distance between two pressure measurement locations of the first flow section, d_{U} is an axial distance between two pressure measurement locations of the second flow section, g is an acceleration due to gravity, θ_{L} is an inclination angle of a flow axis of the first flow section from horizontal, θ_{U} is an inclination angle of the second flow section from horizontal, and ρₘ is a density of the fluid flow; and
determine a water-cut using the following equation:
$$WC = \frac{{ρ}_{m} - {ρ}_{o}}{{ρ}_{w} - {ρ}_{o}}$$
where ρₒ is a density of an oil portion of the fluid flow, ρ_{w} is a water density of the fluid flow, and WC is the water-cut.

5. The downhole production system of claim 1, wherein the first differential pressure sub-system comprises a first pressure sensor and a second pressure sensor, wherein the second differential pressure sub-system comprises a third pressure sensor and a fourth pressure sensor.

6. A method comprising:
receiving a fully developed, multi-phase fluid flow into a measurement device;
directing the fluid flow through a first flow passage of the measurement device;
measuring a pressure drop of the fully developed, multi-phase fluid flow within the first flow passage;
directing the fluid flow from the first flow passage to a second flow passage of the measurement device, the second flow passage fluidically coupled to the first flow passage, the second flow passage being at an angle from the first flow passage;
measuring a pressure drop of the fully developed, multi-phase fluid flow within the second flow passage; and
determining a density of the fluid flow in response to measuring the pressure drop within the first flow passage and the pressure drop within the second flow passage.

7. The method of claim 6, wherein determining a density of the fluid flow comprises using the following equation:
$${ρ}_{m} = \frac{\frac{{DP}_{U}}{{d}_{U}} - \frac{{DP}_{L}}{{d}_{L}}}{g ( Sin {θ}_{U} - Sin {θ}_{L} )}$$
where DP_{L} is the pressure drop within the first flow passage, DP_{U} is the pressure drop within the second flow passage, a, is an axial distance between two pressure measurement locations of the first flow passage, d_{U} is an axial distance between two pressure measurement locations of the second flow passage, g is an acceleration due to gravity θ_{L} is an inclination angle of a flow axis of the first flow passage from horizontal, θ_{U} is an inclination angle of the second flow passage from horizontal, and ρₘ is a density of the fluid flow.

8. The method of claim 6, further comprising determining a water-cut in response to determining the density of the fluid flow.

9. The method of claim 8, wherein determining the water-cut comprises using the following equation:
$$WC = \frac{{ρ}_{m} - {ρ}_{o}}{{ρ}_{w} - {ρ}_{o}}$$
where ρₒ is a density of an oil portion of the fluid flow, ρ_{w} is a water density of the fluid flow, ρₘ is a density of the fluid flow, and WC is the water-cut.

10. A measurement device comprising:
a first flow passage configured to receive a fully developed, multi-phase fluid flow;
a second flow passage configured to receive the fully developed, multi-phase fluid flow, the second flow passage positioned downstream of the first flow passage, the second flow passage fluidically coupled to the first flow passage, the second flow passage being at an angle from the first flow passage;
a first pressure sensor positioned within the first flow passage, the first pressure sensor configured to measure a first pressure within the first flow passage;
a second pressure sensor positioned within the first flow passage, downstream of the first flow sensor, the second pressure sensor configured to measure a second pressure within the first flow passage;
a third pressure sensor positioned within the second flow passage, the third pressure configured to measure a third pressure within the second flow passage;
a fourth pressure sensor positioned within the second flow passage, downstream of the third pressure sensor, the fourth pressure sensor configured to measure a fourth pressure within the second flow passage; and
a controller configured to receive a first pressure signal from the first pressure sensor, a second pressure signal from the second pressure sensor, a third pressure signal from the third pressure sensor, and a fourth pressure signal from the fourth pressure sensor, the controller configured to determine a density of the fully developed, multi-phase fluid flow at least in part based on the first signal, the second signal, the third signal, and the fourth signal.

11. The measurement device of claim 10, wherein the first flow passage and the second flow passage have the same cross-sectional area.

12. The measurement device of claim 10, wherein a distance between the first pressure sensor and an entrance to the first flow passage is at least five effective diameters of the first flow passage.

13. The measurement device of claim 10, wherein a distance between the second pressure sensor and the second flow passage is at least five effective diameters of the first flow passage.

14. The measurement device of claim 10, wherein a distance between the third pressure sensor and the first flow passage is at least five effective diameters of the first flow passage.

15. The measurement device of claim 10, wherein a distance between the fourth pressure sensor and a downstream obstruction is at least five effective diameters of the second flow passage.

16. The measurement device of claim 10, wherein a distance between the first pressure sensor and the second pressure sensor is at least five effective diameters of the first flow passage.

17. The measurement device of claim 10, wherein a distance between the third pressure sensor and the second pressure sensor is at least ten effective diameters of the second flow passage.

18. The measurement device of claim 10, wherein the angle between the first flow passage and the second flow passage is substantially within five to seven degrees.

19. The measurement device of claim 10, wherein a length of the first flow passage and a length of the second flow passage are substantially within fifteen and twenty effective diameters of the respective flow passages.

20. The measurement device of claim 10, wherein a length of the first flow passage is different from a length of the second flow passage.

21. The measurement device of claim 10, wherein the measurement device is configured to be installed upstream of an electric submersible pump.
